(19) United States
(12) Patent Application Publication
(10) Pub. No.: US 2020/0407794 A1
(43) Pub. Date: Dec. 31, 2020

(54) MOLECULAR TARGETS FOR FETAL NUCLEIC ACID ANALYSIS

(71) Applicant: ChromaCode, Inc., Carlsbad, CA (US)

(72) Inventor: Christopher MACDONALD, Carlsbad, CA (US)

(21) Appl. No.: 16/975,678

(22) PCT Filed: Feb. 27, 2019
(86) PCT No.: PCT/US19/19906

§ 371 Date: Aug. 25, 2020

Related U.S. Application Data
(60) Provisional application No. 62/636,632, filed on Feb. 28, 2018.

(51) Int. Cl. C12Q 1/6883; C12Q 1/6806

(52) U.S. Cl. CPC C12Q 1/6883; C12Q 2600/166; C12Q 1/6806

(57) ABSTRACT

The present disclosure provides methods and compositions for evaluation of nucleic acid size distribution and genetic abnormalities. The disclosed methods may be useful in determining size distribution of nucleic acids in a sample, for example, fetal fraction in a plasma sample. The disclosed methods may be useful in identifying or detecting genetic abnormalities from a subject, for example, fetal aneuploidy (e.g., trisomy 21).

### CROSS-REFERENCE

This application claims the benefit of U.S. Provisional Patent Application No. 62/636,632, filed on Feb. 28, 2018, which application is incorporated herein by reference in its entirety.

### BACKGROUND

Digital PCR (dPCR) is a useful method for detection and quantification of nucleic acid targets. The use of labeled oligonucleotide probes enables specific detection of a target present in a partition (e.g., droplet, microwell). dPCR may be used in a variety of nucleic acid detection methods.

### SUMMARY

Disclosed herein, in some aspects, is a method for analyzing a size distribution of nucleic acids, the method comprising: (A) providing a sample comprising: (i) a first plurality of nucleic acids and a second plurality of nucleic acids, wherein the first plurality of nucleic acids each comprise a first nucleic acid sequence of a given length and the second plurality of nucleic acids each comprise a second nucleic acid sequence longer than the given length; (ii) a first set of paired oligonucleotide primers configured to amplify the first nucleic acid sequence; and (iii) a second set of paired oligonucleotide primers, configured to amplify the second nucleic acid sequence; (B) performing an amplification reaction on (a) the first nucleic acid sequence to generate a first signal and (b) the second nucleic acid sequence to generate a second signal; and (C) determining a ratio of a first value derived from the first signal to a second value derived from the second signal, thereby analyzing the size distribution. In some embodiments, the sample further comprises (iv) a first oligonucleotide probe configured to hybridize to a region of the first nucleic acid sequence and (v) a second oligonucleotide probe configured to hybridize to a region of the second nucleic acid sequence. In some embodiments, the first signal is generated from the first oligonucleotide probe and the second signal is generated from the second oligonucleotide probe. In some embodiments, the sample further comprises an intercalating dye. In some embodiments, the first signal or the second signal is generated from the intercalating dye. In some embodiments, the first signal and the second signal are generated from the intercalating dye. In some embodiments, the intercalating dye is SYBR® Green or EvaGreen®. In some embodiments, the first signal or the second signal is generated by mass spectrometry.

Disclosed herein, in some aspects, is a method for analyzing a size distribution of nucleic acids, the method comprising: (A) providing a sample comprising: (i) a first plurality of nucleic acids and a second plurality of nucleic acids, wherein the first plurality of nucleic acids each comprise a first nucleic acid sequence of a given length and the second plurality of nucleic acids each comprise a second nucleic acid sequence longer than the given length; (ii) a first set of paired amplification oligomers configured to amplify the first nucleic acid sequence; (iii) a second set of paired amplification oligomers, configured to amplify the second nucleic acid sequence; (iv) a first detection probe configured to anneal to a region of the first nucleic acid sequence; and (v) a second detection probe configured to anneal to a region of the second nucleic acid sequence; (B) performing an amplification reaction on (a) the first nucleic acid sequence to generate a first signal from the first detection probe and (b) the second nucleic acid sequence to generate a second signal from the second detection probe; and (C) determining a ratio of a first value derived from the first signal to a second value derived from the second signal, thereby analyzing the size distribution. In some embodiments, the first set of paired amplification oligomers comprises: a first forward amplification oligomer; and a first reverse amplification oligomer. In some embodiments, the first set of paired amplification oligomers comprises: a plurality of first forward amplification oligomers; and a plurality of first reverse amplification oligomers. In some embodiments, each of the plurality of first forward amplification oligomers has a different nucleic acid sequence. In some embodiments, a first forward amplification oligomer of the plurality of first forward amplification oligomers is configured to hybridize to a region of the first sequence. In some embodiments, each of the plurality of first reverse amplification oligomers has a different nucleic acid sequence. In some embodiments, a first reverse amplification oligomer of the plurality of first reverse amplification oligomers is configured to hybridize to a region of the first sequence. In some embodiments, the second set of paired amplification oligomers comprises: a second forward amplification oligomer; and a second reverse amplification oligomer. In some embodiments, the second set of paired amplification oligomers comprises: a plurality of second forward amplification oligomers and a plurality of second reverse amplification oligomers. In some embodiments, each of the plurality of second forward amplification oligomers has a different nucleic acid sequence. In some embodiments, a second forward amplification oligomer of the plurality of second forward amplification oligomers is configured to hybridize to a region of the second sequence. In some embodiments, each of the plurality of second reverse amplification oligomers has a different nucleic acid sequence. In some embodiments, a second reverse amplification oligomer of the plurality of second reverse amplification oligomers is configured to hybridize to a region of the second sequence. In some embodiments, the first value and the second value provide a quantitative ratio measurement corresponding to an abundance of the first plurality of nucleic acids and the second plurality of nucleic acids in the sample. In some embodiments, the first detection probe or the second detection probe comprises a non-target-hybridizing sequence. In some embodiments, the first detection probe or the second detection probe is a hairpin detection probe. In some embodiments, the hairpin detection probe is a molecular beacon or a molecular torch. In some embodiments, the sample comprises: genomic DNA, mRNA, cDNA, or a combination thereof. In some embodiments, the sample is derived from plasma from a pregnant woman. In some embodiments, the sample comprises maternal nucleic acid and fetal nucleic acid. In some embodiments, the first plurality of nucleic acids comprises the fetal nucleic acid and the second plurality of nucleic acids comprises the maternal nucleic acid. In some embodiments, the determining of the ratio provides a fetal fraction. In some embodiments, the sample is from an individual having or suspected of having cancer.

In some embodiments, the first signal and the second signal are generated in a single fluorescence channel. In some embodiments, (B) is performed in at least one partition of a plurality of partitions. In some embodiments, the plurality of partitions is a plurality of droplets. In some embodiments, the plurality of partitions is a plurality of wells. In some embodiments, the second nucleic acid sequence comprises at least a portion of the first nucleic acid sequence. In some embodiments, the second nucleic acid sequence comprises the first nucleic acid sequence. In some embodiments, the amplification reaction comprises polymerase chain reaction (PCR). In some embodiments, the PCR is quantitative PCR (qPCR) or digital PCR (dPCR). In some embodiments, the first detection probe comprises a first detectable label and the second detection probe comprises a second detectable label. In some embodiments, the first detection probe and the second detection probe each further comprise a quencher. In some embodiments, during the amplification reaction, the first detectable label is released from the first detection probe and the second detectable label is released from the second detection probe, thereby generating the first signal and the second signal. In some embodiments, the first detectable label and the second detectable label are each selected from the group consisting of a chemiluminescent label, a fluorescent label, and any combination thereof. In some embodiments, the first signal or the second signal is a chemiluminescent signal, a fluorescent signal, or any combination thereof. In some embodiments, the first detection probe and the second detection probe are TaqMan® detection probes. In some embodiments, the method further comprises comparing the ratio to a reference value. In some embodiments, the comparing identifies the presence or absence of a genetic abnormality in the sample. In some embodiments, the reference value corresponds to a ratio of a third value generated from a third nucleic acid sequence and a fourth value generated from a fourth nucleic acid sequence. In some embodiments, the third nucleic acid sequence and the fourth nucleic acid sequence each correspond to a region of nucleic acid not associated with the genetic abnormality. In some embodiments, the reference value is derived from a plurality of third values generated from a plurality of third nucleic acid sequences and a plurality of fourth values generated from a plurality of fourth nucleic acid sequences. In some embodiments, the plurality of third nucleic acid sequences and the plurality of fourth nucleic acid sequences each correspond to a region of nucleic acid not associated with the genetic abnormality. In some embodiments, the genetic abnormality is a fetal aneuploidy. In some embodiments, the second nucleic acid sequence comprises at least a portion of the first nucleic acid sequence. In some embodiments, the method further comprises comparing the ratio to a reference value. In some embodiments, the first value is a quantity of the first plurality of nucleic acids. In some embodiments, the second value is a quantity of the second plurality of nucleic acids. In some embodiments, the ratio is determined without quantifying the first plurality of nucleic acids and the second plurality of nucleic acids. In some embodiments, the amplification reaction comprises qPCR, wherein the first value is derived from amplification kinetics of the first plurality of nucleic acids. In some embodiments, the amplification reaction comprises qPCR, wherein the second value is derived from amplification kinetics of the second plurality of nucleic acids. In some embodiments, the amplification reaction comprises dPCR, wherein the first value is derived from a number of partitions containing the first nucleic acid sequence. In some embodiments, the amplification reaction comprises dPCR, wherein the second value is derived from a number of partitions containing the second nucleic acid sequence.

In some embodiments, in (A), the sample comprises: (vi) one or more additional pluralities of nucleic acids comprising one or more additional nucleic acid sequences; (vii) one or more additional sets of paired amplification oligomers configured to amplify the one or more additional nucleic acid sequences; and (viii) one or more additional detection probes configured to anneal to a region of the one or more additional nucleic acid sequences; in (B), the amplification reaction is performed on the one or more additional nucleic acid sequences to generate one or more additional signals from the one or more additional sets of detection probes; and in (C), determining an additional ratio of the first value or the second value to one or more additional values derived from the one or more additional signals, thereby analyzing the size distribution. In some embodiments, the one or more additional sets of paired amplification oligomers comprise n amplification oligomers; and the one or more additional sets of detection probes comprise n additional detection probes. In some embodiments, n is an integer between 1 and 30. In some embodiments, the first value is a quantity of the first plurality of nucleic acids. In some embodiments, the second value is a quantity of the second plurality of nucleic acids. In some embodiments, the ratio is determined without quantifying the first plurality of nucleic acids and the second plurality of nucleic acids.

Disclosed herein, in some aspects, is a method for identifying the presence or absence of a fetal aneuploidy, comprising: (A) providing a sample comprising: (i) a plurality of fetal nucleic acids, each comprising a first nucleic acid sequence of a given length; (ii) a plurality of maternal nucleic acids, each comprising a second nucleic acid sequence longer than the given length; (iii) a first set of oligonucleotide primers configured to amplify the first nucleic acid sequence; (iv) a second set of oligonucleotide primers configured to amplify the second nucleic acid sequence; (v) a first oligonucleotide probe configured to hybridize to the first nucleic acid sequence; and (vi) a second oligonucleotide probe configured to hybridize to the second nucleic acid sequence; (B) amplifying (a) the first nucleic acid sequence to generate a first signal from the first oligonucleotide probe and (b) the second nucleic acid sequence to generate a second signal from the second oligonucleotide probe; (C) determining a ratio of a value derived from the first signal to a second value derived from the second signal; and (D) comparing the ratio to a reference value, thereby identifying the presence or absence of the fetal aneuploidy. In some embodiments, the first nucleic acid sequence corresponds to a region of nucleic acid potentially associated with the fetal aneuploidy. In some embodiments, the region comprises a region of chromosome 22, chromosome 21, chromosome 18, chromosome 13, chromosome 9, chromosome 8, or an X chromosome. In some embodiments, the region comprises a region of chromosome 21. In some embodiments, the region comprises a region of chromosome 18. In some embodiments, the region comprises a region of chromosome 13. In some embodiments, the region comprises a region of an X chromosome. In some embodiments, the reference value corresponds to a ratio of a third value generated from a third nucleic acid sequence and a fourth value generated from a fourth nucleic acid sequence. In some embodiments, the third nucleic acid sequence and the fourth nucleic acid sequence each correspond to a region of nucleic acid not associated with the fetal aneuploidy. In some embodiments, the reference value is derived from a plurality of third values generated from a plurality of third nucleic acid sequences and a plurality of fourth values generated from a plurality of fourth nucleic acid sequences. In some embodiments, the plurality of third nucleic acid sequences and the plurality of fourth nucleic acid sequences each correspond to a region of nucleic acid not associated with the fetal aneuploidy. In some embodiments, the region is a region of a housekeeping gene. In some embodiments, the housekeeping gene is β-globin. In some embodiments, the ratio is larger than the reference value, thereby indicating the presence of the fetal aneuploidy. In some embodiments, the ratio is smaller than the reference value, thereby identifying the presence of the fetal aneuploidy. In some embodiments, the plurality of fetal nucleic acids and the plurality of maternal nucleic acids are obtained from plasma from a pregnant woman. In some embodiments, the plurality of fetal nucleic acids comprises fetal deoxyribonucleic acid (DNA) and the plurality of maternal nucleic acids comprises maternal DNA. In some embodiments, the amplifying in (b) comprises polymerase chain reaction (PCR). In some embodiments, the PCR is quantitative PCR (qPCR) or digital PCR (dPCR). In some embodiments, the first oligonucleotide probe comprises a first detectable label and the second oligonucleotide probe comprises a second detectable label. In some embodiments, the first oligonucleotide probe and the second oligonucleotide probe each further comprise a quencher. In some embodiments, during the amplifying, the first detectable label is released from the first oligonucleotide probe and the second detectable label is released from the second oligonucleotide probe, thereby generating the first signal and the second signal. In some embodiments, the first detectable label and the second detectable label are each selected from the group consisting of a chemiluminescent label, a fluorescent label, and any combination thereof. In some embodiments, the first signal or the second signal is a chemiluminescent signal, a fluorescent signal, or any combination thereof. In some embodiments, the first oligonucleotide probe and the second oligonucleotide probe are TaqMan® detection probes. In some embodiments, the first set of oligonucleotide primers comprises a first forward primer and a first reverse primer. In some embodiments, the second set of oligonucleotide primers comprises a second forward primer and a second reverse primer. In some embodiments, the fetal aneuploidy is trisomy 21, trisomy 18, trisomy 13, trisomy 9, or trisomy 8. In some embodiments, the fetal aneuploidy is trisomy 21. In some embodiments, the fetal aneuploidy is trisomy 18. In some embodiments, the fetal aneuploidy is trisomy 13. In some embodiments, the fetal aneuploidy is a sex chromosome aneuploidy. In some embodiments, the sex chromosome aneuploidy is Turner syndrome, Klinefelter syndrome, trisomy X, XXY, or XYY.

In some embodiments, the second nucleic acid sequence does not comprise any of the first nucleic acid sequence. In some embodiments, the second nucleic acid sequence comprises at least a portion of the first nucleic acid sequence. In some embodiments, the second nucleic acid sequence comprises the first nucleic acid sequence. In some embodiments, the reference value corresponds to a ratio of a third value generated from a third nucleic acid sequence and a fourth value generated from a fourth nucleic acid sequence. In some embodiments, the second nucleic acid comprises at least a portion of the first nucleic acid sequence. In some embodiments, the first value is a quantity of the first plurality of nucleic acids. In some embodiments, the second value is a quantity of the second plurality of nucleic acids. In some embodiments, the ratio is determined without quantifying the first plurality of nucleic acids and the second plurality of nucleic acids. In some embodiments, the amplification reaction comprises qPCR, wherein the first value is derived from amplification kinetics of the first plurality of nucleic acids. In some embodiments, the amplification reaction comprises qPCR, wherein the second value is derived from amplification kinetics of the second plurality of nucleic acids. In some embodiments, the amplification reaction comprises dPCR, wherein the first value is derived from a number of partitions containing the first nucleic acid sequence. In some embodiments, the amplification reaction comprises dPCR, wherein the second value is derived from a number of partitions containing the second nucleic acid sequence.

In some embodiments, in (A), the sample comprises: (vi) one or more additional pluralities of fetal nucleic acids comprising one or more additional first nucleic acid sequences of a given length; (vii) one or more additional pluralities of maternal nucleic acids comprising one or more additional second nucleic acid sequences longer than the given length; (viii) one or more additional first sets of oligonucleotide primers configured to amplify the one or more additional first nucleic acid sequences; (xi) one or more additional second sets of oligonucleotide primers configured to amplify the one or more additional second nucleic acid sequences; (x) one or more additional first oligonucleotide probes configured to anneal to a region of the one or more additional first nucleic acid sequences; and (xi) one or more additional second oligonucleotide probes configured to anneal to a region of the one or more additional second nucleic acid sequences; in (B), the amplification reaction is performed on: the one or more additional first nucleic acid sequences to generate one or more additional first signals from the one or more additional first oligonucleotide probes; and the one or more additional second nucleic acid sequences to generate one or more additional second signals from the one or more additional second oligonucleotide probes; in (C), an additional ratio of one or more additional first values derived from the one or more additional first signals to one or more additional second values derived from the one or more additional second signals is determined; and in (D), the additional ratio is compared to the reference value. In some embodiments, the one or more additional first sets of oligonucleotide primers comprise n oligonucleotide primers; and the one or more additional first oligonucleotide probes comprise n additional detection probes. In some embodiments, the one or more additional second sets of oligonucleotide primers comprise n oligonucleotide primers; and the one or more additional second oligonucleotide probes comprise n additional detection probes. In some embodiments, n is an integer between 1 and 30. In some embodiments, the first value is a quantity of the first plurality of nucleic acids. In some embodiments, the second value is a quantity of the second plurality of nucleic acids. In some embodiments, the ratio is determined without quantifying the first plurality of nucleic acids and the second plurality of nucleic acids. In some embodiments, the plasma is subjected to conditions sufficient to enrich for fetal nucleic acids. In some embodiments, the plasma is not subjected to conditions sufficient to enrich for fetal nucleic acids. In some embodiments, the plasma is subjected to conditions sufficient to enrich for fetal nucleic acids. In some embodiments, the plasma is not subjected to conditions sufficient to enrich for fetal nucleic acids. In some embodiments, the first plurality of nucleic acids and the second plurality of nucleic acids are derived from the same source. In some embodiments, the first plurality of nucleic acids and the second plurality of nucleic acids are derived from different sources.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate an example method for amplifying nucleic acid sequences of different lengths for identifying a differential in nucleic acid size distributions.

FIGS. 2A and 2B illustrate another example method for amplifying nucleic acid sequences of different lengths for identifying a differential in nucleic acid size distributions.

FIG. 3A shows a simulated distribution of fetal fraction in cell free deoxyribonucleic acid (DNA). FIG. 3B shows a Receiver Operating Characteristic (ROC) curve of a simulated digital polymerase chain reaction (dPCR) assay. FIG. 3C shows the true positive (TP) rate for a simulated dPCR assay relative to the fetal fraction in a sample.

FIG. 4 shows ROC curves for a simulated digital PCR assay with target fetal DNA enriched over maternal DNA by 70% (FIG. 4A) or 20% (FIG. 4B).

### DETAILED DESCRIPTION

Polymerase Chain Reaction (PCR) is a method of exponential amplification of specific target nucleic acid in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2 fold with each cycle leading to approximately a billion-fold increase of the target sequence in 30 cycles.

Digital PCR is a process of partitioning a sample containing one or more targets into a plurality of partitions (e.g., wells, droplets, etc.), performing a PCR reaction in each partition, and recording the fluorescence generated by, for example, a target-specific reporter probe. This is generally performed on a digital PCR instrument that measures the fluorescence from each partition in an optical channel through one or more excitation/emission filter sets.

Frequently, the target-specific nucleic acid probe is a short oligonucleotide complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR. The TaqMan® oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

One tool for diagnosing fetal aneuploidy (e.g., trisomy 21) is digital PCR-based noninvasive prenatal screening (NIPS) testing using cell-free fetal DNA sequences isolated from a maternal blood sample. Standard NIPS can often report false negatives and may vary in sensitivity and/or specificity depending on the target. Furthermore, as only 4-10% of DNA in maternal plasma is of fetal origin, existing methods to detect fetal aneuploidy may be limited by the amount of fetal DNA present in a sample. Recognized herein is a need for noninvasive means of accurately detecting, measuring, and evaluating trace amounts of fetal DNA in maternal plasma.

### Definitions

The term “primer,” or “amplification oligomer,” used herein interchangeably, can refer to an oligonucleotide or nucleic acid configured to bind to another nucleic acid and facilitate one or more reactions, for example, transcription, nucleic acid synthesis, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid, or may bind to the non-coding strand of a nucleic acid. A reverse primer may bind to the coding strand of a nucleic acid, or may bind to the non-coding strand of a nucleic acid.

### Overview

Targeted amplification and subsequent differentiation of nucleic acids based on length is a useful tool in molecular diagnostics, especially where a target nucleic acid sequence is present in trace amounts in a sample. The existence of short fragments of nucleic acid may be indicative of various conditions, including disorders resulting from viral, transplant, and/or cancerous ailments. Accordingly, amplifying known target nucleic acid sequences may establish the existence of circulating, short-fragment nucleic acids. Nucleic acids derived from the same source may be used, for example, to confirm the presence of viral and/or cancerous mutations. Nucleic acids from multiple individuals may be used, for example, for transplant and/or pregnancy diagnostics. Described herein are methods for differentiating nucleic acid fragments of varying length, for detection and analysis of nucleic acids of low abundance in a sample (e.g., fetal nucleic acid in a plasma sample).

In one aspect, the present disclosure provides a method for detecting a fetal aneuploidy. Plasma obtained from a pregnant woman will comprise cell-free fragments of both fetal and maternal nucleic acid (e.g., DNA). Maternal nucleic acids in a cell-free sample from a pregnant woman have a higher average fragment length compared with fetal nucleic acids from the same sample. This differential may be utilized to detect fetal aneuploidy with high accuracy. In general, the disclosed methods comprise the use of multiple sets of oligonucleotide primers, each configured to amplify nucleic acid fragments of different length. For example, one set of oligonucleotide primers may be configured to amplify nucleic acid fragments of smaller length (e.g., fetal nucleic acids), and another set of oligonucleotide primers may be configured to amplify fragments of longer length (e.g., maternal nucleic acids). Each set of oligonucleotide primers may be paired with an oligonucleotide probe to generate a signal associated with each set of primers. Oligonucleotide primers and probes designed in this manner can be used to identify a differential in fragment size distribution (e.g., fetal nucleic acid vs. maternal nucleic acid), by identifying the difference between the signals associated with each set of primers. For example, in samples with little to no fetal nucleic acid fraction, the signals generated from the two sets of oligonucleotide primers would be about identical. By contrast, in samples with high fetal nucleic acid fraction, the signal associated with the fetal (i.e., shorter) nucleic acid fragments would be significantly greater than the signal associated with the maternal (i.e., longer) nucleic acid fragments. In addition, this signal differential, or ratio, can be used to identify fetal aneuploidy by comparing the ratio of a test subject with a reference value, such as the ratio from a healthy subject. A significant difference (e.g., increase or decrease) in a measured ratio compared with such a reference value may positively identify a subject as having a fetal aneuploidy.

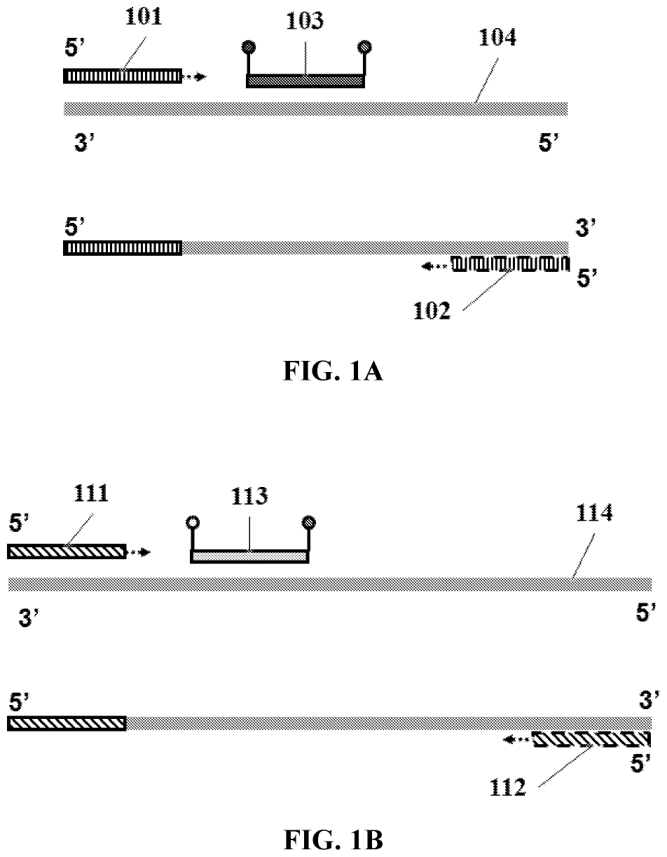

FIGS. 1A and 1B illustrate an example method for targeting different sizes of nucleic acid sequences for nucleic acid analysis. FIG. 1A shows forward primer 101, reverse primer 102, oligonucleotide probe 103, and nucleic acid 104. Forward primer 101 and reverse primer 102 are designed to amplify nucleic acid 104, as shown. Oligonucleotide probe 103 is designed to hybridize to a region of nucleic acid 104, as shown, and is configured to generate a signal following amplification of nucleic acid 104 with forward primer 101 and reverse primer 102. Nucleic acid 104 may be a nucleic acid fragment. Nucleic acid 104 may be a cell-free nucleic acid. Nucleic acid 104 may be a fetal nucleic acid. Nucleic acid 104 is of a given length. For example, nucleic acid 104 may be a cell-free, fetal nucleic acid fragment of a given length. FIG. 1B shows forward primer 111, reverse primer 112, oligonucleotide probe 113, and nucleic acid 114. Forward primer 111 and reverse primer 112 are designed to amplify nucleic acid 114, as shown. Oligonucleotide probe 113 is designed to hybridize to a region of nucleic acid 114, as shown, and is configured to generate a signal following amplification of nucleic acid 114 with forward primer 111 and reverse primer 112. Nucleic acid 114 may be a nucleic acid fragment. Nucleic acid 114 may be a cell-free nucleic acid. Nucleic acid 114 may be a maternal nucleic acid. Nucleic acid 114 is of a length longer than nucleic acid 104. Nucleic acid 114 may comprise a portion of the sequence of nucleic acid 104. Nucleic acid 114 may comprise all of the sequence of nucleic acid 104. Nucleic acid 114 may be of a different sequence than nucleic acid 104. Nucleic acids 104 and 114 may be identified by detection of the signals generated by oligonucleotide probes 103 and 113, respectively, following nucleic acid amplification. A value derived from the signal generated by nucleic acid 104 (e.g., cycle threshold value, partition count, etc.) may be compared to a value derived from the signal generated by nucleic acid 114 (e.g., cycle threshold value, partition count, etc.), thereby generating a ratio. Analysis of this signal ratio may be used, for example, to differentiate nucleic acid 104 from nucleic acid 114 and/or to estimate the size distribution of nucleic acid fragments of varying lengths (e.g., fetal fraction). This ratio can be compared to a reference value, thereby identifying a genetic abnormality (e.g., aneuploidy). For example, the ratio obtained from a subject suspected of having a genetic abnormality can be compared to a ratio obtained from a healthy subject, such that a significant difference in the ratios identifies the subject as having a genetic abnormality.

In some cases, a plurality of different nucleic acids may be analyzed using methods of the present disclosure. For example, a first set of paired oligonucleotide primers may comprise a plurality of forward primers and a plurality of reverse primers, each configured to amplify a nucleic acid sequence of a given length. A first set of paired oligonucleotide primers may be configured to amplify, for example, fetal nucleic acid. A second set of paired oligonucleotide primers may comprise a plurality of forward primers and a plurality of reverse primers, each configured to amplify a nucleic acid sequence of a longer length than the first set of paired oligonucleotide primers. A first set of paired oligonucleotide primers may be configured to amplify, for example, maternal nucleic acid. Signals generated from amplification of fetal nucleic acid and maternal nucleic acid using the first set of paired oligonucleotide primers and the second set of paired oligonucleotide primers may be detected and analyzed, thereby evaluating fetal fraction or identifying a fetal aneuploidy.

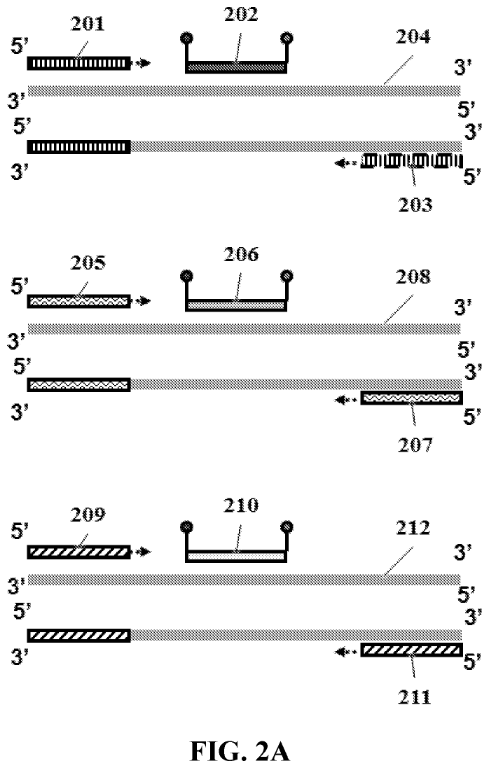

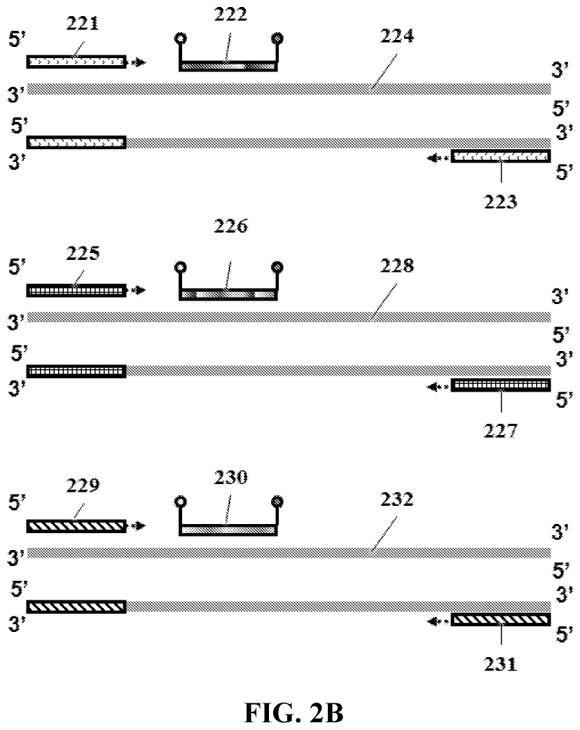

FIGS. 2A and 2B illustrate an example method for targeting different sizes of nucleic acid sequences for nucleic acid analysis. FIG. 2A shows nucleic acids 204, 208, and 212; forward primers 201, 205, and 209; and reverse primers 203, 207, and 211. The forward and reverse primers are each configured to amplify a given nucleic acid, as shown. FIG. 2A also shows oligonucleotide probes 202, 206, and 210, each configured to hybridize to a given nucleic acid and to generate a signal following amplification. Nucleic acids 204, 208, and 212 are each of a given length. Nucleic acids 204, 208, and 212 may be fetal nucleic acid fragments. FIG. 2B shows nucleic acids 224, 228, and 232; forward primers 221, 225, and 229; and reverse primers 223, 227, and 211. The forward and reverse primers are each configured to amplify a given nucleic acid, as shown. FIG. 2B also shows oligonucleotide probes 222, 226, and 230, each configured to hybridize to a given nucleic acid and to generate a signal following amplification. Nucleic acids 224, 228, and 232 are each of a longer length than nucleic acids 204, 208, and 212. Nucleic acids 224, 228, and 232 may be maternal nucleic acid fragments. Nucleic acids 202, 206, 210, 222, 226, and 230 may be identified by detection of signals generated from each of oligonucleotide probes 222, 226, and 230. The signals (e.g., signal intensities) generated by nucleic acids 224, 228, and 232 may be compared to the signals (e.g., signal intensities) generated by nucleic acids 204, 208, and 212, thereby generating a ratio. Analysis of this signal ratio may be used, for example, estimate the size distribution of nucleic acid fragments of varying lengths (e.g., fetal fraction). This ratio can be compared to a reference value, thereby identifying a genetic abnormality (e.g., aneuploidy). For example, the ratio obtained from a subject suspected of having a genetic abnormality can be compared to a ratio obtained from a healthy subject, such that a significant difference in the ratios identifies the subject as having a genetic abnormality.

### Analyzing Nucleic Acid Size Distribution

In some aspects, disclosed herein are methods for analyzing nucleic acid size distribution. First, a sample may be provided comprising: (i) a first plurality of nucleic acids and a second plurality of nucleic acids, wherein the first plurality of nucleic acids each comprise a first nucleic acid sequence of a given length and the second plurality of nucleic acids each comprise a second nucleic acid sequence longer than the given length; (ii) a first set of paired amplification oligomers configured to amplify the first nucleic acid sequence; (iii) a second set of paired amplification oligomers, configured to amplify the second nucleic acid sequence; (iv) a first detection probe configured to anneal to a region of the first nucleic acid sequence; and (v) a second detection probe configured to anneal to a region of the second nucleic acid sequence. In some cases, a first detection probe and a second detection probe are not provided. Next, amplification may be performed on the first nucleic acid sequence and the second nucleic acid sequence. The amplification may generate a first signal from the first detection probe and a second signal from the second detection probe. Alternatively, in cases where a first detection probe and a second detection probe are not provided, the amplification may generate a first signal and/or a second signal from an intercalating dye (e.g., SYBR® Green, EvaGreen®) and/or from mass spectrometry. Next, a ratio of a value derived from the first signal to value derived from the second signal may be determined, thereby analyzing nucleic acid size distribution.

The first set of paired oligonucleotide primers may comprise a first forward primer and a first reverse primer (i.e., a first pair of oligonucleotide primers). A first pair of oligonucleotide primers (e.g., a first forward primer and a first reverse primer) may be configured to amplify a nucleic acid sequence of a given length (e.g., may hybridize to regions of a nucleic acid sequence a given distance apart). A first pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A first pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less. A first pair of oligonucleotide primers may be configured to amplify a nucleic acid sequence of a length of about 50, about 75, about 100, about 125, about 150, about 175, about 200, about 225, about 250, about 275, or about 300 bp. In some cases, a first pair of oligonucleotide primers is configured to amplify a nucleic acid sequence of a length of about 70 bp. In some cases, a first pair of oligonucleotide primers is configured to amplify a nucleic acid sequence of a length of about 100 bp. In some cases, a first pair of oligonucleotide primers is configured to amplify a nucleic acid sequence of a length of about 150 bp. The first set of paired oligonucleotide primers may comprise a plurality of first forward primers and a plurality of first reverse primers (i.e., a first plurality of paired oligonucleotide primers). The first set of paired oligonucleotide primers may comprise at least 2, at least 3, at least 3, at least 4, at least 5, at least 6, at least 7, at least 8, at least 9, or at least 10 pairs of oligonucleotide primers, or more. The first set of paired oligonucleotide primers may comprise about 2, about 3, about 3, about 4, about 5, about 6, about 7, about 8, about 9, or about 10 pairs of oligonucleotide primers. The first set of paired oligonucleotide primers may comprise n pairs of oligonucleotide primers. n may be an integer. n may be an integer from 2-30. n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. n may be an integer greater than 30. Each pair of oligonucleotide primers in a first set of paired oligonucleotide primers may be configured to amplify a nucleic acid sequence of a given length. In some cases, each pair of oligonucleotide primers in a first set of paired oligonucleotide primers is configured to amplify a nucleic acid sequence of about the same length (e.g., about 70 bp, about 100 bp, about 150 bp, or more). In some cases, some or all of the pairs of oligonucleotide primers in a first set of paired oligonucleotide primers are configured to amplify a nucleic acid sequence of a different length. For example, a pair of oligonucleotide primers in a first set of oligonucleotide primers may be configured to amplify a nucleic acid sequence of a length of about 70 bp, and another pair of oligonucleotide primers in a first set of oligonucleotide primers may be configured to amplify a nucleic acid sequence of a length of about 100 bp.

The second set of paired oligonucleotide primers may comprise a second forward primer and a second reverse primer (i.e., a second pair of oligonucleotide primers). A second pair of oligonucleotide primers (e.g., a second forward primer and a second reverse primer) may be configured to amplify a nucleic acid sequence of a given length (e.g., may hybridize to regions of a nucleic acid sequence a given distance apart). A second pair of oligonucleotide primers may be configured to amplify a nucleic acid sequence of a length longer than a nucleic acid sequence amplified by a first pair of oligonucleotide primers. A second pair of oligonucleotide primers may be configured to amplify a nucleic acid sequence of a length of at least 300, at least 325, at least 350, at least 375, at least 400, at least 425, at least 450, at least 500, at least 550, at least 600, at least 650, at least 700, or at least 750 base pairs (bp), or more. A second pair of oligonucleotide primers may be configured to amplify a nucleic acid sequence of a length of at most 750, at most 700, at most 650, at most 600, at most 550, at most 500, at most 750, at most 425, at most 400, at most 375, at most 350, at most 325, at most 350, at most 325, or at most 300 bp, or less. A second pair of oligonucleotide primers may be configured to amplify a nucleic acid sequence of a length of about 300, about 325, about 350, about 375, about 400, about 425, about 450, about 500, about 550, about 600, about 650, about 700, or about 750 bp. In some cases, a second pair of oligonucleotide primers is configured to amplify a nucleic acid sequence of a length of about 300 bp. In some cases, a second pair of oligonucleotide primers is configured to amplify a nucleic acid sequence of a length of about 500 bp. In some cases, a second pair of oligonucleotide primers is configured to amplify a nucleic acid sequence of a length of about 750 bp. The second set of paired oligonucleotide primers may comprise a plurality of second forward primers and a plurality of second reverse primers (i.e., a second plurality of paired oligonucleotide primers). The second set of paired oligonucleotide primers may comprise at least 2, at least 3, at least 3, at least 4, at least 5, at least 6, at least 7, at least 8, at least 9, or at least 10 pairs of oligonucleotide primers, or more. The second set of paired oligonucleotide primers may comprise about 2, about 3, about 3, about 4, about 5, about 6, about 7, about 8, about 9, or about 10 pairs of oligonucleotide primers. The second set of paired oligonucleotide primers may comprise n pairs of oligonucleotide primers. n may be an integer. n may be an integer from 2-30. n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. n may be an integer greater than 30. Each pair of oligonucleotide primers in a second set of paired oligonucleotide primers may be configured to amplify a nucleic acid sequence of a given length. Each pair of oligonucleotide primers in a second set of paired oligonucleotide primers may be configured to amplify a nucleic acid sequence of a length longer than a nucleic acid sequence amplified by a first set of paired oligonucleotide primers. In some cases, each pair of oligonucleotide primers in a second set of paired oligonucleotide primers is configured to amplify a nucleic acid sequence of about the same length (e.g., about 300 bp, about 500 bp, about 750 bp, or more). In some cases, some or all of the pairs of oligonucleotide primers in a second set of paired oligonucleotide primers are each configured to amplify a nucleic acid sequence of a different length. For example, a pair of oligonucleotide primers in a second set of oligonucleotide primers may be configured to amplify a nucleic acid sequence of a length of about 300 bp, and another pair of oligonucleotide primers in a second set of oligonucleotide primers may be configured to amplify a nucleic acid sequence of a length of about 500 bp.

The first detection probe or the second detection probe may comprise a non-target hybridizing sequence. A non-target hybridizing sequence may be a region which is not complementary to any target nucleic acid. The first detection probe or the second detection probe may be a molecular beacon. The first detection probe or the second detection probe may be a molecular torch. The first detection probe or the second detection probe may be a molecular beacon. The first detection probe or the second detection probe may comprise a detectable label. A detectable label may be a chemiluminescent label. A detectable label may be a fluorescent label. The first detection probe and the second detection probe may each comprise a different detectable label. For example, the first detection probe may comprise a fluorophore of a first color, and the second detection probe may comprise a fluorophore of a second color. The first detection probe and the second detection probe may each comprise an identical detectable label. The first detection probe and the second detection probe may each comprise a quencher. The first detection probe and the second detection probe may be TaqMan® detection probes.

The amplification may be linear amplification. The amplification may comprise polymerase chain reaction (PCR). The amplification may be digital PCR. The amplification may be quantitative PCR. The amplification may be performed in a partition of a plurality of partitions. The amplification may be performed in a droplet in an emulsion. The amplification may be performed in a microwell.

Determining the ratio of the first value to the second value may provide a fetal fraction. The ratio may be compared to a reference value. Comparing the ratio to the reference value may determine an estimated fetal fraction in a sample. Comparing the ratio to the reference value may identify the presence or absence of a genetic abnormality (e.g., an aneuploidy) in a sample. A reference value may correspond to a ratio of a third value generated from a third nucleic acid sequence and a fourth value generated from a fourth nucleic acid sequence. The third nucleic acid sequence and the fourth nucleic acid sequence may each correspond to a region of nucleic acid not associated with a genetic abnormality.

The first plurality of nucleic acids may be a plurality of fetal nucleic acids. The second plurality of nucleic acids may be a plurality of maternal nucleic acids. The first nucleic acid sequence may correspond to a region of a fetal nucleic acid associated with a fetal aneuploidy. The region associated with a fetal aneuploidy may be, for example, a region of chromosome 21, a region of chromosome 18, a region of chromosome 13, or a region of an X chromosome.

A method for analyzing nucleic acid size distribution may comprise providing one or more additional pluralities of nucleic acids comprising one or more additional nucleic acid sequences, one or more additional sets of amplification oligomers, and one or more additional detection probes. One or more additional sets of amplification oligomers may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional sets of amplification oligomers. One or more additional sets of amplification oligomers may be n additional sets of amplification oligomers. One or more additional pluralities of nucleic acids may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional pluralities of nucleic acids. One or more additional pluralities of nucleic acids may be n additional pluralities of nucleic. One or more additional detection probes may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional detection probes. One or more additional detection probes may be n additional detection probes. n may be an integer. n may be an integer between 1 and 30. n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. n may be an integer greater than 30.

### Detecting Fetal Aneuploidy

In some aspects, disclosed herein are methods for identifying the presence or absence of a fetal aneuploidy. First, a sample may be provided comprising: (i) a plurality of fetal nucleic acids comprising a sequence of a given length; (ii) a plurality of maternal nucleic acids comprising a sequence longer than the given length; (iii) a first set of oligonucleotide primers configured to amplify the first nucleic acid sequence; (iv) a second set of oligonucleotide primers, configured to amplify the second nucleic acid sequence; (v) a first oligonucleotide probe configured to anneal to a region of the first nucleic acid sequence; and (vi) a second oligonucleotide probe configured to anneal to a region of the second nucleic acid sequence. In some cases, a first oligonucleotide probe and a second oligonucleotide probe are not provided. Next, amplification may be performed on the first nucleic acid sequence and the second nucleic acid sequence. The amplification may generate a first signal from the first oligonucleotide probe and a second signal from the second oligonucleotide probe. Alternatively, in cases where a first oligonucleotide probe and a second oligonucleotide probe are not provided, the amplification may generate a first signal and/or a second signal from an intercalating dye (e.g., SYBR® Green, EvaGreen®) and/or from mass spectrometry. Next, a ratio of a value derived from the first signal to a value generated from the second signal may be determined. Next, the ratio may be compared to a reference value, thereby identifying the presence of absence of the fetal aneuploidy.

The first set of paired oligonucleotide primers may comprise a first forward primer and a first reverse primer (i.e., a first pair of oligonucleotide primers). A first pair of oligonucleotide primers (e.g., a first forward primer and a first reverse primer) may be configured to amplify a nucleic acid sequence of a given length (e.g., may hybridize to regions of a nucleic acid sequence a given distance apart). A first pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A first pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less. A first pair of oligonucleotide primers may be configured to amplify a nucleic acid sequence of a length of about 50, about 75, about 100, about 125, about 150, about 175, about 200, about 225, about 250, about 275, or about 300 bp. In some cases, a first pair of oligonucleotide primers is configured to amplify a nucleic acid sequence of a length of about 70 bp. In some cases, a first pair of oligonucleotide primers is configured to amplify a nucleic acid sequence of a length of about 100 bp. In some cases, a first pair of oligonucleotide primers is configured to amplify a nucleic acid sequence of a length of about 150 bp. The first set of paired oligonucleotide primers may comprise a plurality of first forward primers and a plurality of first reverse primers (i.e., a first plurality of paired oligonucleotide primers). The first set of paired oligonucleotide primers may comprise at least 2, at least 3, at least 3, at least 4, at least 5, at least 6, at least 7, at least 8, at least 9, or at least 10 pairs of oligonucleotide primers, or more. The first set of paired oligonucleotide primers may comprise about 2, about 3, about 3, about 4, about 5, about 6, about 7, about 8, about 9, or about 10 pairs of oligonucleotide primers. Each pair of oligonucleotide primers in a first set of paired oligonucleotide primers may be configured to amplify a nucleic acid sequence of a given length. In some cases, each pair of oligonucleotide primers in a first set of paired oligonucleotide primers is configured to amplify a nucleic acid sequence of about the same length (e.g., about 70 bp, about 100 bp, about 150 bp, or more). In some cases, some or all of the pairs of oligonucleotide primers in a first set of paired oligonucleotide primers are each configured to amplify a nucleic acid sequence of a different length. For example, a pair of oligonucleotide primers in a first set of oligonucleotide primers may be configured to amplify a nucleic acid sequence of a length of about 70 bp, and another pair of oligonucleotide primers in a first set of oligonucleotide primers may be configured to amplify a nucleic acid sequence of a length of about 100 bp.

The second set of paired oligonucleotide primers may comprise a second forward primer and a second reverse primer (i.e., a second pair of oligonucleotide primers). A second pair of oligonucleotide primers (e.g., a second forward primer and a second reverse primer) may be configured to amplify a nucleic acid sequence of a given length (e.g., may hybridize to regions of a nucleic acid sequence a given distance apart). A second pair of oligonucleotide primers may be configured to amplify a nucleic acid sequence of a length longer than a nucleic acid sequence amplified by a first pair of oligonucleotide primers. A second pair of oligonucleotide primers may be configured to amplify a nucleic acid sequence of a length of at least 300, at least 325, at least 350, at least 375, at least 400, at least 425, at least 450, at least 500, at least 550, at least 600, at least 650, at least 700, or at least 750 base pairs (bp), or more. A second pair of oligonucleotide primers may be configured to amplify a nucleic acid sequence of a length of at most 750, at most 700, at most 650, at most 600, at most 550, at most 500, at most 750, at most 425, at most 400, at most 375, at most 350, at most 325, at most 350, at most 325, or at most 300 bp, or less. A second pair of oligonucleotide primers may be configured to amplify a nucleic acid sequence of a length of about 300, about 325, about 350, about 375, about 400, about 425, about 450, about 500, about 550, about 600, about 650, about 700, or about 750 bp. In some cases, a second pair of oligonucleotide primers is configured to amplify a nucleic acid sequence of a length of about 300 bp. In some cases, a second pair of oligonucleotide primers is configured to amplify a nucleic acid sequence of a length of about 500 bp. In some cases, a second pair of oligonucleotide primers is configured to amplify a nucleic acid sequence of a length of about 750 bp. The second set of paired oligonucleotide primers may comprise a plurality of second forward primers and a plurality of second reverse primers (i.e., a second plurality of paired oligonucleotide primers). The second set of paired oligonucleotide primers may comprise at least 2, at least 3, at least 3, at least 4, at least 5, at least 6, at least 7, at least 8, at least 9, or at least 10 pairs of oligonucleotide primers, or more. The second set of paired oligonucleotide primers may comprise about 2, about 3, about 3, about 4, about 5, about 6, about 7, about 8, about 9, or about 10 pairs of oligonucleotide primers. Each pair of oligonucleotide primers in a second set of paired oligonucleotide primers may be configured to amplify a nucleic acid sequence of a given length. Each pair of oligonucleotide primers in a second set of paired oligonucleotide primers may be configured to amplify a nucleic acid sequence of a length longer than a nucleic acid sequence amplified by a first set of paired oligonucleotide primers. In some cases, each pair of oligonucleotide primers in a second set of paired oligonucleotide primers is configured to amplify a nucleic acid sequence of about the same length (e.g., about 300 bp, about 500 bp, about 750 bp, or more). In some cases, some or all of the pairs of oligonucleotide primers in a second set of paired oligonucleotide primers are configured to amplify a nucleic acid sequence of a different length. For example, a pair of oligonucleotide primers in a second set of oligonucleotide primers may be configured to amplify a nucleic acid sequence of a length of about 300 bp, and another pair of oligonucleotide primers in a second set of oligonucleotide primers may be configured to amplify a nucleic acid sequence of a length of about 500 bp.

The plurality of fetal nucleic acids and the plurality of maternal nucleic acids may be obtained from plasma from a pregnant woman. The fetal nucleic acids may be fetal deoxyribonucleic acids (DNA). The maternal nucleic acids may be maternal DNA. The first nucleic acid sequence may correspond to a region of a fetal nucleic acid associated with a fetal aneuploidy. The region associated with a fetal aneuploidy may be, for example, a region of chromosome 21, a region of chromosome 18, a region of chromosome 13, or a region of an X chromosome.

The ratio may be larger than the reference value, thereby indicating the presence of a fetal aneuploidy. The ratio may be smaller than the reference value, thereby indicating the presence of a fetal aneuploidy. The ratio may be about the same as the reference value, thereby indicating the absence of a fetal aneuploidy. The reference value may correspond to a ratio of a third value generated from a third nucleic acid sequence and a fourth value generated from a fourth nucleic acid sequence. A third nucleic acid sequence and a fourth nucleic acid sequence may each correspond to a region of nucleic acid not associated with a fetal aneuploidy. A region of nucleic acid not associated with fetal aneuploidy may be a region of a housekeeping gene. A housekeeping gene may be, for example, β-globin.

The first oligonucleotide probe or the second oligonucleotide probe may comprise a non-target hybridizing sequence. A non-target hybridizing sequence may be a region which is not complementary to any target nucleic acid. The first oligonucleotide probe or the second oligonucleotide probe may be a molecular beacon. The first oligonucleotide probe or the second oligonucleotide probe may be a molecular torch. The first oligonucleotide probe or the second oligonucleotide probe may be a molecular beacon. The first oligonucleotide probe or the second oligonucleotide probe may comprise a detectable label. A detectable label may be a chemiluminescent label. A detectable label may be a fluorescent label. The first oligonucleotide probe and the second oligonucleotide probe may each comprise a different detectable label. For example, the first oligonucleotide probe may comprise a fluorophore of a first color, and the second oligonucleotide probe may comprise a fluorophore of a second color. The first oligonucleotide probe and the second oligonucleotide probe may each comprise an identical detectable label. The first oligonucleotide probe and the second oligonucleotide probe may each comprise a quencher. The first detection probe and the second oligonucleotide probe may be TaqMan® oligonucleotide probes.

The amplification may be linear amplification. The amplification may comprise polymerase chain reaction (PCR). The amplification may be digital PCR. The amplification may be quantitative PCR. The amplification may be performed in a partition of a plurality of partitions. The amplification may be performed in a droplet in an emulsion. The amplification may be performed in a microwell.

A fetal aneuploidy may be an abnormal number of chromosomes. The fetal aneuploidy may be a trisomy. A trisomy may be trisomy 21. A trisomy may be trisomy 18. A trisomy may be trisomy 13. The fetal aneuploidy may be a monosomy. A monosomy may be Turner syndrome. A fetal aneuploidy may be a sex chromosome aneuploidy. A sex chromosome aneuploidy may be, for example, XO, XXX, XXY, or XYY.

A method for analyzing nucleic acid size distribution may comprise providing one or more additional pluralities of nucleic acids comprising one or more additional nucleic acid sequences, one or more additional sets of amplification oligomers, and one or more additional detection probes. One or more additional sets of amplification oligomers may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional sets of amplification oligomers. One or more additional sets of amplification oligomers may be n additional sets of amplification oligomers. One or more additional pluralities of nucleic acids may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional pluralities of nucleic acids. One or more additional pluralities of nucleic acids may be n additional pluralities of nucleic. One or more additional detection probes may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional detection probes. One or more additional detection probes may be n additional detection probes. n may be an integer. n may be an integer between 1 and 30. n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. n may be an integer greater than 30.

### Genetic Abnormalities

The disclosed methods may be used in diagnosing, detecting, or otherwise identifying one or more genetic abnormalities from a nucleic acid sample. The disclosed methods may be used in a test that diagnoses, detects, or otherwise identifies one or more genetic abnormalities from a nucleic acid sample, where data obtained from methods described herein (e.g., nucleic acid size distribution, estimated fetal fraction, etc.) is used to aid the detection or filter out samples unlikely to result in an accurate result. A genetic abnormality may be, for example, a chromosomal abnormality (e.g., chromosomal translocation, aneuploidy, etc), a genetic mutation (e.g., insertion, deletion, etc.), or a nucleic acid variant (e.g., single nucleotide polymorphism). In some cases, a genetic abnormality identified by the disclosed methods is an aneuploidy. An aneuploidy may be a fetal aneuploidy. In some cases, the disclosed methods comprise amplification of nucleic acid sequences from a chromosome associated with a fetal aneuploidy. Chromosomes associated with fetal aneuploidy include, for example, chromosome 21 (e.g., associated with trisomy 21), chromosome 18 (e.g., associated with trisomy 18), chromosome 13 (e.g., associated with trisomy 13), and an X chromosome (e.g., associated with sex chromosome aneuploidies, for example, Turner syndrome, Klinefelter syndrome, trisomy X, XXY, XYY, etc.).

An aneuploidy may describe the presence of an abnormal number of chromosomes in a sample or subject (e.g., a fetus). An aneuploidy may be a trisomy. In some cases, a trisomy identified via the disclosed methods is trisomy 21 (i.e., Down syndrome). In some cases, a trisomy identified via the disclosed methods is trisomy 13. In some cases, a trisomy identified via the disclosed methods is trisomy 13. In some cases, a trisomy identified via the disclosed methods is trisomy X. In some cases, a trisomy identified via the disclosed methods is XYY. In some cases, a trisomy identified via the disclosed methods is Klinefelter syndrome. An aneuploidy may be a monosomy. In some cases, a monosomy identified via the disclosed methods is monosomy X (i.e., Turner syndrome).

### Oligonucleotide Primers

An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine. An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence of a given length. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence of given length under conditions sufficient for nucleic acid amplification. Different sets of oligonucleotide primers may be configured to amplify nucleic acid sequences of different lengths. In one example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A set of paired oligonucleotide primers may comprise a forward primer and a reverse primer (i.e., a pair of oligonucleotide primers). A pair of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less. A pair of oligonucleotide primers may be configured to amplify a nucleic acid sequence of a length of about 50, about 75, about 100, about 125, about 150, about 175, about 200, about 225, about 250, about 275, or about 300 bp. In some cases, a pair of oligonucleotide primers is configured to amplify a nucleic acid sequence of a length of about 70 bp. In some cases, a pair of oligonucleotide primers is configured to amplify a nucleic acid sequence of a length of about 100 bp. In some cases, a pair of oligonucleotide primers is configured to amplify a nucleic acid sequence of a length of about 150 bp. A pair of oligonucleotide primers may be configured to amplify a nucleic acid sequence of a length of at least 300, at least 325, at least 350, at least 375, at least 400, at least 425, at least 450, at least 500, at least 550, at least 600, at least 650, at least 700, or at least 750 base pairs (bp), or more. A pair of oligonucleotide primers may be configured to amplify a nucleic acid sequence of a length of at most 750, at most 700, at most 650, at most 600, at most 550, at most 500, at most 750, at most 425, at most 400, at most 375, at most 350, at most 325, at most 350, at most 325, or at most 300 bp, or less. A pair of oligonucleotide primers may be configured to amplify a nucleic acid sequence of a length of about 300, about 325, about 350, about 375, about 400, about 425, about 450, about 500, about 550, about 600, about 650, about 700, or about 750 bp. In some cases, a pair of oligonucleotide primers is configured to amplify a nucleic acid sequence of a length of about 300 bp. In some cases, a pair of oligonucleotide primers is configured to amplify a nucleic acid sequence of a length of about 500 bp. In some cases, a pair of oligonucleotide primers is configured to amplify a nucleic acid sequence of a length of about 750 bp. A set of paired oligonucleotide primers may comprise a plurality of forward primers and a plurality of reverse primers (i.e., a plurality of paired oligonucleotide primers). A set of paired oligonucleotide primers may comprise at least 2, at least 3, at least 3, at least 4, at least 5, at least 6, at least 7, at least 8, at least 9, or at least 10 pairs of oligonucleotide primers, or more. A set of paired oligonucleotide primers may comprise about 2, about 3, about 3, about 4, about 5, about 6, about 7, about 8, about 9, or about 10 pairs of oligonucleotide primers. Each pair of oligonucleotide primers in a set of paired oligonucleotide primers may be configured to amplify a nucleic acid sequence of a given length. In some cases, each pair of oligonucleotide primers in a set of paired oligonucleotide primers is configured to amplify a nucleic acid sequence of about the same length (e.g., about 70 bp, about 100 bp, about 150 bp, about 300 bp, about 500 bp, about 750 bp or more). In some cases, some or all of the pairs of oligonucleotide primers in a set of paired oligonucleotide primers are each configured to amplify a nucleic acid sequence of a different length. For example, a pair of oligonucleotide primers in a set of oligonucleotide primers may be configured to amplify a nucleic acid sequence of a length of about 70 bp, and another pair of oligonucleotide primers in a set of oligonucleotide primers may be configured to amplify a nucleic acid sequence of a length of about 100 bp. Nucleic acid enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe.

### Thermal Cycling and Amplification

Methods of the present disclosure may comprise thermal cycling. Thermal cycling may comprise one or more thermal cycles. Thermally cycling may be performed under reaction conditions sufficient to amplify one or more nucleic acids. Sufficient reaction conditions may include sufficient temperature conditions, sufficient buffer conditions, and the presence of sufficient reagents. Sufficient temperature conditions may be such that each thermal cycle is performed at a desired annealing temperature. A desired annealing temperature may be sufficient for annealing of a region of an oligonucleotide primer to a target nucleic acid. A desired annealing temperature may be sufficient for annealing of a region of an oligonucleotide probe to a target nucleic acid. Sufficient buffer conditions may be such that the required salts are present in a buffer used during thermal cycling. Required salts may include magnesium salts, potassium salts, and/or ammonium salts. Sufficient buffer conditions may be such that the appropriate salts are present at required concentrations. Sufficient reagents for amplification of nucleic acids with PCR may include deoxytriphosphates (dNTPs). dNTPs may comprise natural or non-natural dNTPs including, for example, dATP, dCTP, dGTP, dTTP, dUTP, and variants thereof.

Amplification of nucleic acid targets may be performed using a suitable amplification method. Amplification may comprise linear amplification. Amplification may comprise polymerase chain reaction (PCR). Amplification may comprise a nucleic acid extension reaction. Amplification may be performed before or after partitioning nucleic acids. In some cases, amplification of nucleic acids is performed in a plurality of partitions. For example, nucleic acid targets may be partitioned into a plurality of droplets, and amplification performed within each droplet.

### Signal Generation and Detection

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme. A signal may be a chemiluminescent signal. A signal may be a fluorescent signal. A detectable label may comprise a quencher and a fluorophore, such that the quencher is released from a detectable label upon degradation of an oligonucleotide probe, thereby generating a fluorescent signal. Thermal cycling of the present disclosure may generate a signal. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be generated by detectable labels comprising the same fluorophore. Detectable labels comprising the same fluorophore may generate different signals by nature of being at different concentrations, thereby generating different intensities of the same signal type. A signal may be generated by an intercalating dye (e.g., SYBR® Green, EvaGreen®). A signal may be generated by mass spectrometry (e.g., may be a mass spectrum corresponding to a nucleic acid).

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. In some cases, a signal intensity increases with each thermal cycle. A signal intensity may increase in a sigmoidal fashion. A signal may be generated and used to derive (e.g., calculate) a cycle threshold value. A signal may be generated and used to derive (e.g., calculate) a partition count.

### Partitioning

Methods of the present disclosure may comprise partitioning nucleic acids, oligonucleotide probes, and, in some cases, additional reagents into a plurality of partitions. A partition may be a droplet (e.g., a droplet in an emulsion). A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids.

### Oligonucleotide Probes

Samples, mixtures, kits, and compositions of the present disclosure may comprise an oligonucleotide probe, also referenced herein as a detection probe. An oligonucleotide probe may be a nucleic acid (e.g., DNA, RNA, etc.). An oligonucleotide probe may comprise a region complementary to a region of a target nucleic acid. The concentration of an oligonucleotide probe may be such that it is in excess relative to other components in a sample. A sample may comprise more than one oligonucleotide probe. Multiple oligonucleotide probes may be the same, or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length.

An oligonucleotide probe may comprise a non-target-hybridizing sequence. A non-target-hybridizing sequence may be a sequence which is not complementary to any region of a target nucleic acid sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

An oligonucleotide probe may comprise a detectable tag. A detectable label may be a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Target Nucleic Acids

A nucleic acid of the present disclosure may be derived from any source including, for example, viruses, bacterial cells, and eukaryotic cells. A nucleic acid may be derived from one or more cells. A cell may be a tumor cell. A cell may be a cell suspected of comprising a viral pathogen. In some cases, a nucleic acid is derived from a cell-free sample (e.g., serum, plasma). Nucleic acid may be derived from plasma of a subject. For example, nucleic acid (e.g., maternal nucleic acid and fetal nucleic acid) may be derived from a plasma sample of a pregnant woman. A nucleic acid may be cell-free nucleic acid. Cell-free nucleic acid may be, for example, cell-free tumor DNA, cell-free fetal DNA, cell-free RNA, etc. A nucleic acid may comprise deoxyribonucleic acid (DNA). DNA may be any kind of DNA, including genomic DNA. A nucleic acid may be viral DNA. A nucleic acid may comprise ribonucleic acid (RNA). RNA may be any kind of RNA, including messenger RNA, transfer RNA, ribosomal RNA, and microRNA. RNA may be viral RNA. A nucleic acid may comprise a gene whose detection may be useful in diagnosing one or more diseases. A gene may be a viral gene or bacterial gene whose detection may be useful in identifying the presence or absence of a pathogen in a subject.

A nucleic acid may be a fetal nucleic acid. A nucleic acid may be a maternal nucleic acid. A nucleic acid may comprise one or more nucleic acid sequences which may be detected or amplified by the methods of the present disclosure. A nucleic acid sequence may correspond to a region of a nucleic acid potentially associated with an aneuploidy. For example, a nucleic acid sequence of a fetal nucleic acid may be associated with a fetal aneuploidy. A nucleic acid sequence may be a region of a chromosome associated with a fetal aneuploidy. Chromosomes associated with fetal aneuploidy include, for example, chromosome 21 (e.g., associated with trisomy 21), chromosome 18 (e.g., associated with trisomy 18), chromosome 13 (e.g., associated with trisomy 13), and an X chromosome (e.g., associated with sex chromosome aneuploidies, for example, Turner syndrome, Klinefelter syndrome, and trisomy X). In some cases, the methods of the present disclosure are useful in identifying and estimating a fetal fraction in a sample. In some cases, the methods of the present disclosure are useful in identifying the presence or absence of a fetal aneuploidy in a sample. In some cases, the methods of the present disclosure are useful in detecting the presence or absence or one or more infectious agents (e.g., viruses) in a subject. In some cases, the methods of the present disclosure are useful in detecting the relative amount of a fetal nucleic acid in a cell-free nucleic acid sample from a subject, thereby diagnosing the fetus for one or more genetic abnormalities (e.g., fetal aneuploidy). In some cases, the methods of the present disclosure are useful in detecting the presence or absence of tumor DNA in a cell-free nucleic acid sample from a subject, thereby diagnosing the subject for cancer.

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample comprising nucleic acids may be processed to enrich for fetal nucleic acid. A sample comprising nucleic acids may be processed to enrich for nucleic acid fragments smaller than a given size. A sample may be enriched for nucleic acid of interest (e.g., fetal nucleic acid) by various methods including, for example, by size exclusion filtration, sequence-specific enrichment (e.g., via use of capture sequences), epigenetic-specific enrichment (e.g., via use of methylation-specific capture moieties, such as antibodies). Enrichment may comprise isolation of nucleic acid of interest and/or depletion of nucleic acid that is not of interest. In some cases, a sample is not processed to purify or enrich for nucleic acid of interest prior to performing methods of the present disclosure (e.g., amplification of nucleic acids from a sample). For example, a sample may not be processed to enrich for fetal nucleic acid prior to mixing a sample with oligonucleotide primers and oligonucleotide probes, as described elsewhere herein. The disclosed methods may be capable of, for example, identifying fetal fraction and/or identifying a fetal aneuploidy regardless of whether a sample has been purified or enriched for fetal nucleic acid.

### Kits

Also provided herein are kits useful in, for example, analyzing nucleic acid size distribution (e.g., fetal fraction) and/or identifying the presence or absence of a fetal aneuploidy in a sample. Kits may comprise one or more oligonucleotide probes. Oligonucleotide probes may be lyophilized. Different oligonucleotide probes may be present at different concentrations in a kit. Oligonucleotide probes may comprise a detectable label, which may comprise, for example, a fluorophore and one or more quenchers.

Kits may comprise one or more sets of oligonucleotide primers (or “amplification oligomers”) as described herein. A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence of a given length. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence of given length under conditions sufficient for nucleic acid amplification. Different sets of oligonucleotide primers may be configured to amplify nucleic acid sequences of different lengths. In one example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence. Oligonucleotide primers configured to amplify sequences of different lengths may be provided together in a kit, which may be used in performing the disclosed methods (e.g., size distribution analysis, fetal aneuploidy detection, etc.). Oligonucleotide primers may be lyophilized. In some cases, all of the oligonucleotide primers may be lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. Kits may comprise instructions for using any of the foregoing in the methods described herein.

### EXAMPLES

### Example 1—Simulated Digital PCR Assay for Fetal Nucleic Acid Analysis

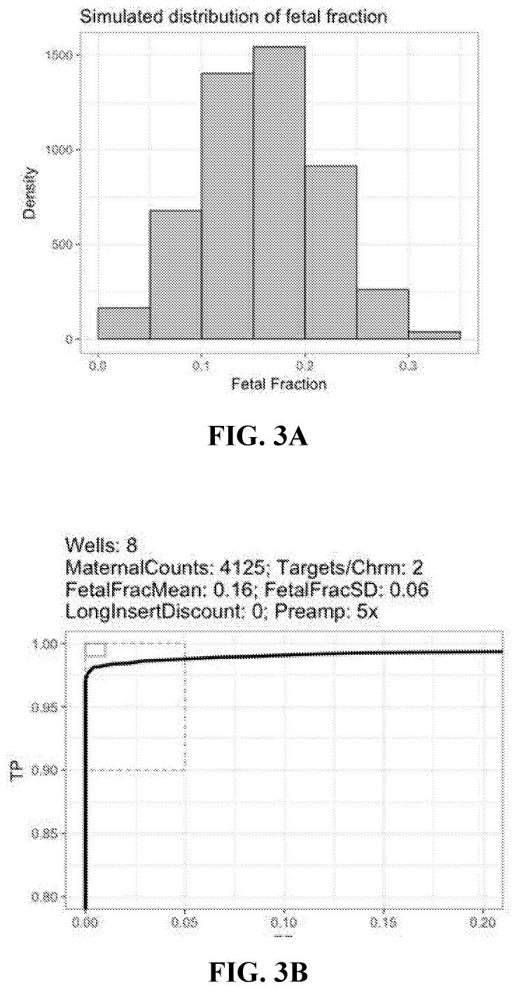

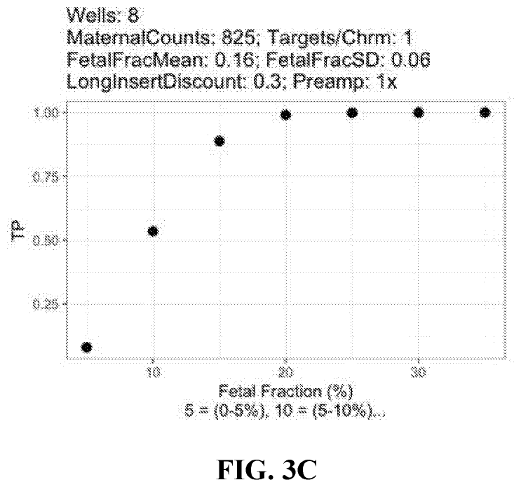

Current attempts at using digital PCR to detect fetal aneuploidy (e.g., trisomy 21) or other fetal conditions can be limited by the amount of fetal DNA present in a sample. FIG. 3A shows a simulated distribution of fetal fraction in cell free deoxyribonucleic acid (DNA). To evaluate the sensitivity and specificity of digital PCR assays, a simulated digital PCR for detection of fetal aneuploidy was generated. This simulated assay contains 8 wells, an average of 4,125 maternal counts of DNA, two targets per chromosome and a mean fetal fraction of 0.16 with a standard deviation of 0.06. FIG. 3B shows a Receiver Operating Characteristic (ROC) curve for the simulated assay, showing true positive (TP) rate versus false positive (FP) rate. The region within the dotted line represents the area with >90% true positive rate and <5% false positive rate. The area within the solid line represents area with >99% true positive rate and <1% false positive rate. This data demonstrates limited sensitivity, driven primarily by samples with low fetal fraction. FIG. 3C shows the true positive (TP) rate for the simulated digital PCR assay of FIG. 3B, relative to the fetal fraction in a sample. These data demonstrate that the success rate of a simulated digital PCR assay for detection of fetal aneuploidy is highly dependent on the fetal fraction of the sample.

### Example 2—Simulated Digital PCR Assay for Fetal Nucleic Acid Analysis Using a Cutoff Filter

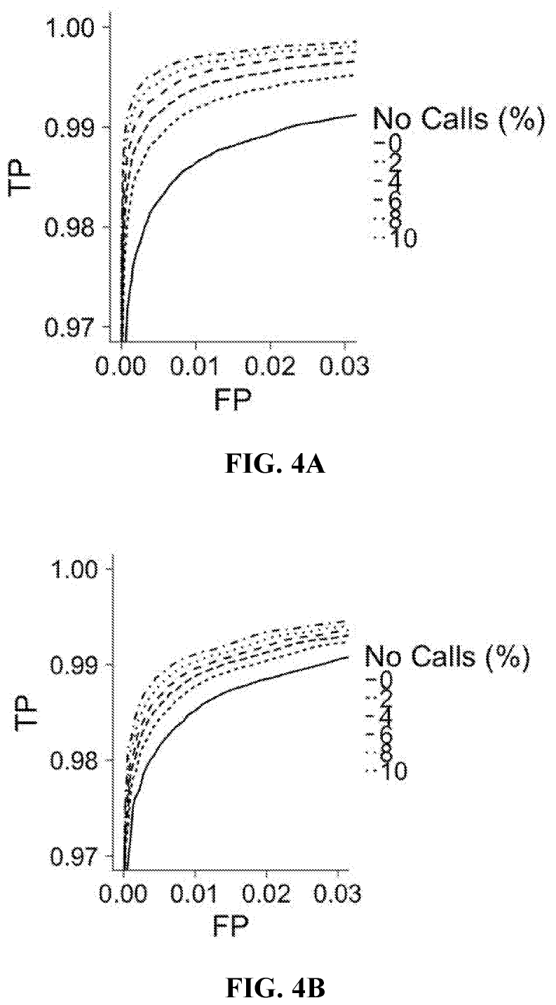

To evaluate the sensitivity and specificity of a digital PCR assay using a cutoff filter for low fetal fraction samples, a simulated digital PCR for detection of fetal aneuploidy was generated. The simulation consisted of a Monte Carlo approach consisting of trials of distributing the input maternal counts of DNA and randomly chosen fetal fraction according to the following distribution. DNA counts were then randomly partitioned into 20,000 virtual partitions in two wells to simulate the performance of digital PCR instrumentation. This simulated assay contains 2 wells, an average of 4,125 maternal counts of DNA, ten targets per chromosome, and a mean fetal fraction of 0.16 with a standard deviation of 0.06. FIG. 4A shows an ROC curve for the simulated assay with a target enriched in maternal DNA by 70% relative to a housekeeping gene. Here, a cutoff filter was used to filter out low accuracy samples with low fetal fraction. The cutoff filter was varied such that it excluded a given percentage of the simulated trials, as shown in FIG. 4A and FIG. 4B. By increasing the amount of “No Calls” using such a filter, significant increases in sensitivity and specificity of the simulated assay were observed. FIG. 4B shows an ROC curve for the simulated assay with a target enriched in maternal DNA by 20% relative to a housekeeping gene, using a cutoff filter as in FIG. 4A. Even with only a 20% difference in fetal DNA, increasing the amount of “No Calls” using a filter resulted in significant gains in specificity and sensitivity of the simulated assay.

### Example 3—Determining Fetal Fraction Using Two Pairs of Oligonucleotide Primers

A plasma sample from a pregnant woman is obtained. The plasma sample comprises fetal cell-free nucleic acid and maternal cell-free nucleic acid. The fetal and maternal nucleic acid are mixed with a pair of oligonucleotide primers and a second pair of oligonucleotide primers, each specific for a sequence on chromosome 21. The first pair of oligonucleotide primers is designed to amplify a nucleic acid sequence from chromosome 21 of a length of about 100 base pairs (bp). The second pair of oligonucleotide primers is designed to amplify a nucleic acid sequence from chromosome 21 of a length of about 300 bp. Also provided are first and second TaqMan® oligonucleotide probes, corresponding to the first and second pairs of oligonucleotide primers, designed for detecting each nucleic acid sequence.

The fetal and maternal nucleic acid are subjected to droplet digital polymerase chain reaction using the provided oligonucleotide primers and probes. A signal corresponding to each nucleic acid sequence from chromosome 21 is detected. A quantity of nucleic acid is derived from each signal. The ratio of the quantity derived from the signal generated from the first pair of oligonucleotide primers to the quantity derived from the signal generated from the second pair of oligonucleotide primers is calculated. This ratio is compared to a reference ratio obtained from signals similarly generated by amplification of nucleic acid sequences from β-globin. The ratio of signal generated from chromosome 21 is higher than the reference ratio, thereby identifying the sample as containing a fetal fraction. The comparison is used to calculate an estimated fetal fraction in the sample.

### Example 4—Determining Fetal Fraction Using Two Sets of Oligonucleotide Primers Each Comprising Two Pairs of Oligonucleotide Primers

A plasma sample from a pregnant woman is obtained. The plasma sample comprises fetal cell-free nucleic acid and maternal cell-free nucleic acid. The fetal and maternal nucleic acid are mixed with a first set of paired oligonucleotide primers and a second set of paired oligonucleotide primers. Each set comprises two pairs of oligonucleotide primers (i.e., two forward primers, each paired with a reverse primer). Each pair of oligonucleotide primers in the first and second set is specific for a different sequence on chromosome 21. The first set of paired oligonucleotide primers comprises two pairs of oligonucleotide primers, each designed to amplify a different nucleic acid sequence from chromosome 21 of a length of about 70 base pairs (bp). The second set of paired oligonucleotide primers comprises two pairs of oligonucleotide primers, each designed to amplify a different nucleic acid sequence from chromosome 21 of a length of about 500 bp. Also provided are a first set of TaqMan® oligonucleotide probes and second set of TaqMan® oligonucleotide probes designed for detecting each nucleic acid sequence. Each set of TaqMan® oligonucleotide probes comprises two oligonucleotide probes corresponding to the two primer pairs of each of the first and second sets of oligonucleotide primers.

The fetal and maternal nucleic acid are subjected to droplet digital polymerase chain reaction using the provided oligonucleotide primers and probes. A signal corresponding to each nucleic acid sequence from chromosome 21 is detected. A quantity of nucleic acid is derived from each signal. The ratio of the quantity derived from the signals generated from the first set of oligonucleotide primers to the quantity derived from the signals generated from the second set of oligonucleotide primers is calculated. This ratio is compared to a reference ratio obtained from signals similarly generated by amplification of nucleic acid sequences from β-globin. The ratio of signal generated from chromosome 21 is higher than the reference ratio, thereby identifying the sample as containing a fetal fraction. The comparison is used to calculate an estimated fetal fraction in the sample.

The term “about” when referring to a measurable value such as an amount, a temporal duration, and the like, is meant to encompass variations of 20% or in some instances 10%, or in some instances ±5%, or in some instances ±1%, or in some instances 0.1% from the specified value, as such variations are appropriate to perform the disclosed methods. Further, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art. About also includes the exact amount. Hence “about 200 nM” means “about 200 nM” and also “200 nM”.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for analyzing a size distribution of nucleic acids, the method comprising:
(A) providing a sample comprising:
(i) a first plurality of nucleic acids and a second plurality of nucleic acids, wherein said first plurality of nucleic acids each comprise a first nucleic acid sequence of a given length and said second plurality of nucleic acids each comprise a second nucleic acid sequence longer than said given length;
(ii) a first set of paired amplification oligomers configured to amplify said first nucleic acid sequence;
(iii) a second set of paired amplification oligomers, configured to amplify said second nucleic acid sequence;
(iv) a first detection probe configured to anneal to a region of said first nucleic acid sequence; and
(v) a second detection probe configured to anneal to a region of said second nucleic acid sequence;
(B) performing an amplification reaction on (a) said first nucleic acid sequence to generate a first signal from said first detection probe and (b) said second nucleic acid sequence to generate a second signal from said second detection probe; and
(C) determining a ratio of a first value derived from said first signal to a second value derived from said second signal, thereby analyzing said size distribution.

2. The method of claim 1, wherein said first set of paired amplification oligomers comprises: a first forward amplification oligomer; and a first reverse amplification oligomer.

3. The method of claim 2, wherein said first set of paired amplification oligomers comprises: a plurality of first forward amplification oligomers; and a plurality of first reverse amplification oligomers.

4. The method of claim 3, wherein each of said plurality of first forward amplification oligomers has a different nucleic acid sequence.

5. The method of claim 4, wherein a first forward amplification oligomer of said plurality of first forward amplification oligomers is configured to hybridize to a region of said first sequence.

6. The method of claim 3, wherein each of said plurality of first reverse amplification oligomers has a different nucleic acid sequence.

7. The method of claim 6, wherein a first reverse amplification oligomer of said plurality of first reverse amplification oligomers is configured to hybridize to a region of said first sequence.

8. The method of claim 1, wherein said second set of paired amplification oligomers comprises: a second forward amplification oligomer; and a second reverse amplification oligomer.

9. The method of claim 8, wherein said second set of paired amplification oligomers comprises: a plurality of second forward amplification oligomers and a plurality of second reverse amplification oligomers.

10. The method of claim 9, wherein each of said plurality of second forward amplification oligomers has a different nucleic acid sequence.

11. The method of claim 10, wherein a second forward amplification oligomer of said plurality of second forward amplification oligomers is configured to hybridize to a region of said second sequence.

12. The method of claim 9, wherein each of said plurality of second reverse amplification oligomers has a different nucleic acid sequence.

13. The method of claim 12, wherein a second reverse amplification oligomer of said plurality of second reverse amplification oligomers is configured to hybridize to a region of said second sequence.

14. The method of claim 1, wherein said first value and said second value provide a quantitative ratio measurement corresponding to an abundance of said first plurality of nucleic acids and said second plurality of nucleic acids in said sample.

15. The method of any of claims 1-14, wherein said first detection probe or said second detection probe comprises a non-target-hybridizing sequence.

16. The method of claim 15, wherein said first detection probe or said second detection probe is a hairpin detection probe.

17. The method of claim 16, wherein said hairpin detection probe is a molecular beacon or a molecular torch.

18. The method of any of claims 1-17, wherein said sample comprises: genomic DNA, mRNA, cDNA, or a combination thereof.

19. The method of any of claims 1-18, wherein said sample is derived from plasma from a pregnant woman.

20. The method of claim 19, wherein said sample comprises maternal nucleic acid and fetal nucleic acid.

21. The method of claim 20, wherein said first plurality of nucleic acids comprises said fetal nucleic acid and wherein said second plurality of nucleic acids comprises said maternal nucleic acid.

22. The method of claim 20 or 21, wherein said determining of said ratio provides a fetal fraction.

23. The method of any of claims 1-22, wherein said sample is from an individual having or suspected of having cancer.

24. The method of any of claims 1-23, wherein said first signal and said second signal are generated in a single fluorescence channel.

25. The method of any of claims 1-24, wherein (B) is performed in at least one partition of a plurality of partitions.

26. The method of claim 25, wherein said plurality of partitions is a plurality of droplets.

27. The method of claim 25, wherein said plurality of partitions is a plurality of wells.

28. The method of claim 1, wherein said second nucleic acid sequence comprises at least a portion of said first nucleic acid sequence.

29. The method of claim 28, wherein said second nucleic acid sequence comprises said first nucleic acid sequence.

30. The method of any of claims 1-29, wherein said amplification reaction comprises polymerase chain reaction (PCR).

31. The method of claim 30, wherein said PCR is quantitative PCR (qPCR) or digital PCR (dPCR).

32. The method of any of claims 1-31, wherein said first detection probe comprises a first detectable label and said second detection probe comprises a second detectable label.

33. The method of claim 32, wherein said first detection probe and said second detection probe each further comprise a quencher.

34. The method of claim 32 or 33, wherein, during said amplification reaction, said first detectable label is released from said first detection probe and said second detectable label is released from said second detection probe, thereby generating said first signal and said second signal.

35. The method of any of claims 32-34, wherein said first detectable label and said second detectable label are each selected from the group consisting of a chemiluminescent label, a fluorescent label, and any combination thereof.

36. The method of claim 35, wherein said first signal or said second signal is a chemiluminescent signal, a fluorescent signal, or any combination thereof.

37. The method of claim 32-36, wherein said first detection probe and said second detection probe are TaqMan® detection probes.

38. The method of claim 1, further comprising comparing said ratio to a reference value.

39. The method of claim 38, wherein said comparing identifies the presence or absence of a genetic abnormality in said sample.

40. The method of claim 39, wherein said reference value corresponds to a ratio of a third value generated from a third nucleic acid sequence and a fourth value generated from a fourth nucleic acid sequence.

41. The method of claim 40, wherein said third nucleic acid sequence and said fourth nucleic acid sequence each correspond to a region of nucleic acid not associated with said genetic abnormality.

42. The method of claim 41, wherein said reference value is derived from a plurality of third values generated from a plurality of third nucleic acid sequences and a plurality of fourth values generated from a plurality of fourth nucleic acid sequences.

43. The method of claim 42, wherein said plurality of third nucleic acid sequences and said plurality of fourth nucleic acid sequences each correspond to a region of nucleic acid not associated with said genetic abnormality.

44. The method of any of claims 39-43, wherein said genetic abnormality is a fetal aneuploidy.

45. The method of any of claims 1-27, wherein said second nucleic acid sequence comprises at least a portion of said first nucleic acid sequence.

46. The method of any of claims 1-38, further comprising comparing said ratio to a reference value.

47. The method of any of claims 1-46, wherein said first value is a quantity of said first plurality of nucleic acids.

48. The method of any of claims 1-47, wherein said second value is a quantity of said second plurality of nucleic acids.

49. The method of any of claims 1-46, wherein said ratio is determined without quantifying said first plurality of nucleic acids and said second plurality of nucleic acids.

50. The method of any of claims 1-49, wherein said amplification reaction comprises qPCR, wherein said first value is derived from amplification kinetics of said first plurality of nucleic acids.

51. The method of any of claims 1-49, wherein said amplification reaction comprises qPCR, wherein said second value is derived from amplification kinetics of said second plurality of nucleic acids.

52. The method of any of claims 1-49, wherein said amplification reaction comprises dPCR, wherein said first value is derived from a number of partitions containing said first nucleic acid sequence.

53. The method of any of claims 1-49, wherein said amplification reaction comprises dPCR, wherein said second value is derived from a number of partitions containing said second nucleic acid sequence.

54. The method of any of claims 1-53, wherein:
in (A), said sample comprises:
(vi) one or more additional pluralities of nucleic acids comprising one or more additional nucleic acid sequences;
(vii) one or more additional sets of paired amplification oligomers configured to amplify said one or more additional nucleic acid sequences; and
(viii) one or more additional detection probes configured to anneal to a region of said one or more additional nucleic acid sequences;
in (B), said amplification reaction is performed on said one or more additional nucleic acid sequences to generate one or more additional signals from said one or more additional sets of detection probes; and
in (C), determining an additional ratio of said first value or said second value to one or more additional values derived from said one or more additional signals, thereby analyzing said size distribution.

55. The method of claim 54, wherein: said one or more additional sets of paired amplification oligomers comprise n amplification oligomers; and said one or more additional sets of detection probes comprise n additional detection probes.

56. The method of claim 55, wherein n is an integer between 1 and 30.

57. The method of claim 1, wherein said first value is a quantity of said first plurality of nucleic acids.

58. The method of claim 1, wherein said second value is a quantity of said second plurality of nucleic acids.

59. The method of claim 1, wherein said ratio is determined without quantifying said first plurality of nucleic acids and said second plurality of nucleic acids.

60. A method for identifying the presence or absence of a fetal aneuploidy, comprising:
(A) providing a sample comprising:
(i) a plurality of fetal nucleic acids, each comprising a first nucleic acid sequence of a given length;
(ii) a plurality of maternal nucleic acids, each comprising a second nucleic acid sequence longer than said given length;
(iii) a first set of oligonucleotide primers configured to amplify said first nucleic acid sequence;
(iv) a second set of oligonucleotide primers configured to amplify said second nucleic acid sequence;
(v) a first oligonucleotide probe configured to hybridize to said first nucleic acid sequence; and
(vi) a second oligonucleotide probe configured to hybridize to said second nucleic acid sequence;
(B) amplifying (a) said first nucleic acid sequence to generate a first signal from said first oligonucleotide probe and (b) said second nucleic acid sequence to generate a second signal from said second oligonucleotide probe;
(C) determining a ratio of a value derived from said first signal to a second value derived from said second signal; and
(D) comparing said ratio to a reference value, thereby identifying said presence or absence of said fetal aneuploidy.

61. The method of claim 60, wherein said first nucleic acid sequence corresponds to a region of nucleic acid potentially associated with said fetal aneuploidy.

62. The method of claim 61, wherein said region comprises a region of chromosome 22, chromosome 21, chromosome 18, chromosome 13, chromosome 9, chromosome 8, or an X chromosome.

63. The method of claim 61, wherein said region comprises a region of chromosome 21.

64. The method of claim 62, wherein said region comprises a region of chromosome 18.

65. The method of claim 62, wherein said region comprises a region of chromosome 13.

66. The method of claim 62, wherein said region comprises a region of an X chromosome.

67. The method of claim 60, wherein said reference value corresponds to a ratio of a third value generated from a third nucleic acid sequence and a fourth value generated from a fourth nucleic acid sequence.

68. The method of claim 67, wherein said third nucleic acid sequence and said fourth nucleic acid sequence each correspond to a region of nucleic acid not associated with said fetal aneuploidy.

69. The method of claim 60, wherein said reference value is derived from a plurality of third values generated from a plurality of third nucleic acid sequences and a plurality of fourth values generated from a plurality of fourth nucleic acid sequences.

70. The method of claim 69, wherein said plurality of third nucleic acid sequences and said plurality of fourth nucleic acid sequences each correspond to a region of nucleic acid not associated with said fetal aneuploidy.

71. The method of claim 67-70, wherein said region is a region of a housekeeping gene.

72. The method of claim 71, wherein said housekeeping gene is β-globin.

73. The method of any of claims 60-72, wherein said ratio is larger than said reference value, thereby indicating the presence of said fetal aneuploidy.

74. The method of any of claims 60-72, wherein said ratio is smaller than said reference value, thereby identifying the presence of said fetal aneuploidy.

75. The method of any of claims 60-74, wherein said plurality of fetal nucleic acids and said plurality of maternal nucleic acids are obtained from plasma from a pregnant woman.

76. The method of any of claims 60-75, wherein said plurality of fetal nucleic acids comprises fetal deoxyribonucleic acid (DNA) and said plurality of maternal nucleic acids comprises maternal DNA.

77. The method of any of claims 60-76, wherein said amplifying in (b) comprises polymerase chain reaction (PCR).

78. The method of claim 77, wherein said PCR is quantitative PCR (qPCR) or digital PCR (dPCR).

79. The method of any of claims 60-78, wherein said first oligonucleotide probe comprises a first detectable label and said second oligonucleotide probe comprises a second detectable label.

80. The method of claim 79, wherein said first oligonucleotide probe and said second oligonucleotide probe each further comprise a quencher.

81. The method of claim 79 or 80, wherein, during said amplifying, said first detectable label is released from said first oligonucleotide probe and said second detectable label is released from said second oligonucleotide probe, thereby generating said first signal and said second signal.

82. The method of any of claims 79-81, wherein said first detectable label and said second detectable label are each selected from the group consisting of a chemiluminescent label, a fluorescent label, and any combination thereof.

83. The method of claim 82, wherein said first signal or said second signal is a chemiluminescent signal, a fluorescent signal, or any combination thereof.

84. The method of any of claims 60-83, wherein said first oligonucleotide probe and said second oligonucleotide probe are TaqMan® detection probes.

85. The method of any of claims 60-84, wherein said first set of oligonucleotide primers comprises a first forward primer and a first reverse primer.

86. The method of any of claims 60-85, wherein said second set of oligonucleotide primers comprises a second forward primer and a second reverse primer.

87. The method of any of claims 60-86, wherein said fetal aneuploidy is trisomy 21, trisomy 18, trisomy 13, trisomy 9, or trisomy 8.

88. The method of claim 87, wherein said fetal aneuploidy is trisomy 21.

89. The method of claim 87, wherein said fetal aneuploidy is trisomy 18.

90. The method of claim 87, wherein said fetal aneuploidy is trisomy 13.

91. The method of claim 87, wherein said fetal aneuploidy is a sex chromosome aneuploidy.

92. The method of claim 91, wherein said sex chromosome aneuploidy is Turner syndrome, Klinefelter syndrome, trisomy X, XXY, or XYY.

93. The method of any of claims 60-92, wherein said second nucleic acid sequence does not comprise any of said first nucleic acid sequence.

94. The method of claim 60, wherein said second nucleic acid sequence comprises at least a portion of said first nucleic acid sequence.

95. The method of claim 94, wherein said second nucleic acid sequence comprises said first nucleic acid sequence.

96. The method of any of claims 60-66, wherein said reference value corresponds to a ratio of a third value generated from a third nucleic acid sequence and a fourth value generated from a fourth nucleic acid sequence.

97. The method of any of claims 60-92, wherein said second nucleic acid comprises at least a portion of said first nucleic acid sequence.

98. The method of any of claims 60-97, wherein said first value is a quantity of said first plurality of nucleic acids.

99. The method of any of claims 60-98, wherein said second value is a quantity of said second plurality of nucleic acids.

100. The method of any of claims 60-97, wherein said ratio is determined without quantifying said first plurality of nucleic acids and said second plurality of nucleic acids.

101. The method of any of claims 60-100, wherein said amplification reaction comprises qPCR, wherein said first value is derived from amplification kinetics of said first plurality of nucleic acids.

102. The method of any of claims 60-100, wherein said amplification reaction comprises qPCR, wherein said second value is derived from amplification kinetics of said second plurality of nucleic acids.

103. The method of any of claims 60-100, wherein said amplification reaction comprises dPCR, wherein said first value is derived from a number of partitions containing said first nucleic acid sequence.

104. The method of any of claims 60-100, wherein said amplification reaction comprises dPCR, wherein said second value is derived from a number of partitions containing said second nucleic acid sequence.

105. The method of any of claims 60-104, wherein:
in (A), said sample comprises:
(vi) one or more additional pluralities of fetal nucleic acids comprising one or more additional first nucleic acid sequences of a given length;
(vii) one or more additional pluralities of maternal nucleic acids comprising one or more additional second nucleic acid sequences longer than said given length;
(viii) one or more additional first sets of oligonucleotide primers configured to amplify said one or more additional first nucleic acid sequences;
(xi) one or more additional second sets of oligonucleotide primers configured to amplify said one or more additional second nucleic acid sequences;
(x) one or more additional first oligonucleotide probes configured to anneal to a region of said one or more additional first nucleic acid sequences; and
(xi) one or more additional second oligonucleotide probes configured to anneal to a region of said one or more additional second nucleic acid sequences;
in (B), said amplification reaction is performed on: said one or more additional first nucleic acid sequences to generate one or more additional first signals from said one or more additional first oligonucleotide probes; and said one or more additional second nucleic acid sequences to generate one or more additional second signals from said one or more additional second oligonucleotide probes;
in (C), an additional ratio of one or more additional first values derived from said one or more additional first signals to one or more additional second values derived from said one or more additional second signals is determined; and
in (D), said additional ratio is compared to said reference value.

106. The method of claim 105, wherein: said one or more additional first sets of oligonucleotide primers comprise n oligonucleotide primers; and said one or more additional first oligonucleotide probes comprise n additional detection probes.

107. The method of claim 105, wherein: said one or more additional second sets of oligonucleotide primers comprise n oligonucleotide primers; and said one or more additional second oligonucleotide probes comprise n additional detection probes.

108. The method of claim 106 or 107, wherein n is an integer between 1 and 30.

109. The method of claim 60, wherein said first value is a quantity of said first plurality of nucleic acids.

110. The method of claim 60, wherein said second value is a quantity of said second plurality of nucleic acids.

111. The method of claim 60, wherein said ratio is determined without quantifying said first plurality of nucleic acids and said second plurality of nucleic acids.

112. The method of claim 19, wherein said plasma is subjected to conditions sufficient to enrich for fetal nucleic acids.

113. The method of claim 19, wherein said plasma is not subjected to conditions sufficient to enrich for fetal nucleic acids.

114. The method of claim 59, wherein said plasma is subjected to conditions sufficient to enrich for fetal nucleic acids.

115. The method of claim 59, wherein said plasma is not subjected to conditions sufficient to enrich for fetal nucleic acids.

116. The method of any of claims 1-60, wherein said first plurality of nucleic acids and said second plurality of nucleic acids are derived from the same source.

117. The method of any of claims 1-60, wherein said first plurality of nucleic acids and said second plurality of nucleic acids are derived from different sources.

118. A method for analyzing a size distribution of nucleic acids, the method comprising:
(A) providing a sample comprising:
(i) a first plurality of nucleic acids and a second plurality of nucleic acids, wherein said first plurality of nucleic acids each comprise a first nucleic acid sequence of a given length and said second plurality of nucleic acids each comprise a second nucleic acid sequence longer than said given length;
(ii) a first set of paired oligonucleotide primers configured to amplify said first nucleic acid sequence; and
(iii) a second set of paired oligonucleotide primers, configured to amplify said second nucleic acid sequence;
(B) performing an amplification reaction on (a) said first nucleic acid sequence to generate a first signal and (b) said second nucleic acid sequence to generate a second signal; and
(C) determining a ratio of a first value derived from said first signal to a second value derived from said second signal, thereby analyzing said size distribution.

119. The method of claim 118, wherein said sample further comprises (iv) a first oligonucleotide probe configured to hybridize to a region of said first nucleic acid sequence and (v) a second oligonucleotide probe configured to hybridize to a region of said second nucleic acid sequence.

120. The method of claim 119, wherein said first signal is generated from said first oligonucleotide probe and said second signal is generated from said second oligonucleotide probe.

121. The method of claim 118, wherein said sample further comprises an intercalating dye.

122. The method of claim 121, wherein said first signal or said second signal is generated from said intercalating dye.

123. The method of claim 121, wherein said first signal and said second signal are generated from said intercalating dye.

124. The method of claim 118, wherein said intercalating dye is SYBR® Green or EvaGreen®.

125. The method of claim 118, wherein said first signal or said second signal are generated by mass spectrometry.
